(19) United States
(12) Patent Application Publication
(10) Pub. No.: US 2018/0028487 A1
(43) Pub. Date: Feb. 1, 2018

(54) DERMATOLOGICAL FORMULATIONS OF 2-(2-ETHOXY-2-OXOETHYL)(METHYL)AMINO-2-OXOETHYL 5-(TETRADECYLOXY)FURAN-2-CARBOXYLATE

(71) Applicant: Dermira Inc., Menlo Park, CA (US)

(72) Inventors: Anthony Adrian Shaw, Menlo Park, CA (US); Kanjai Khumtaveeporn, Aurora (CA); Pavel Krasik, Aurora (CA)

(21) Appl. No.: 15/660,520

(22) Filed: Jul. 26, 2017

Related U.S. Application Data
(60) Provisional application No. 62/366,932, filed on Jul. 26, 2016.

(51) Int. Cl. A61K 31/341; C07D 307/68

(52) U.S. Cl. CPC A61K 31/341; C07D 307/68

(57) ABSTRACT

Disclosed herein are dermatological formulations comprising low-impurity TOFA prodrug 2-(2-ethoxy-2-oxoethyl)(methyl)amino-2-oxoethyl 5-(tetradecyloxy)furan-2-carboxylate represented by Formula (Ia) and pharmaceutical use thereof:

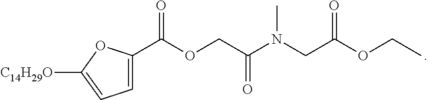

### CROSS REFERENCE TO RELATED APPLICATIONS

This application claims the benefit under 35 U.S.C. §119(e) to U.S. Provisional Application No. 62/366,932, filed Jul. 26, 2016, which application is hereby incorporated by reference in its entirety.

### BACKGROUND

### Technical Field

This disclosure is generally related to dermatological formulations of prodrugs of 5-(tetradecyloxy)-2-furoic acid (TOFA).

### Background

Fatty acid synthesis starts with the carboxylation of acetyl CoA to malonyl CoA. This irreversible reaction is the committed step in fatty acid synthesis. The synthesis of malonyl CoA is catalyzed by acetyl CoA carboxylase (ACC) (See, Brownsey, R. W. et al., “Regulation of acetyl-CoA carboxylase”, Biochem Soc. Trans. (2006) 34: 223-227).

Inhibition of ACC can be effective in diminishing fatty acid synthesis. Long-chain (16-20 carbons) fatty acid acyl-CoA thioesters have been found to be potent physiological end-product inhibitors of mammalian ACC.

TOFA (5-(tetradecyloxy)-2-furoic acid) is a known fatty acid mimetic, which can be converted intracellularly to its acyl-CoA thioester, thus inhibiting ACC activity with a mechanism similar to long chain fatty acid acyl-CoA thioesters. See, McCune, S. A. et al., J. Biol. Chem. (1979), Vol. 254, No. 20, pp. 10095-10101.

TOFA has the following structure:

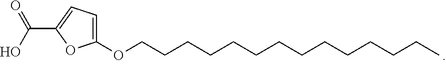 [SMILES: CCCCCCCCCCCCCCOc1ccc(C(=O)O)o1]

TOFA has been shown to reduce plasma triglyceride levels in both rats and monkeys. See, e.g., Parker, R. A. et al., J. Med. Chem. (1977), Vol. 20, pp. 781-791. It has also been known to inhibit hepatic fatty acid synthesis. See, e.g., Ribereau-Gayon, G., FEBS Lett. (1976), Vol. 62, No. 309-312; Panek, E. et al., Lipids (1977), Vol. 12, pp. 814-818; Kariya, T. et al., Biochem. Biophys. Res. Commun. (1978), Vol. 80, pp. 1022-1024; and Harris, R. A. et al., Hormones and Energy Metabolism (Klachko, D. M. et al., eds.), Vol. III, pp. 17-42. TOFA is further known to inhibit sebaceous gland disorders by lowering sebum production. See, e.g., U.S. Published Patent No. 2010/0204317, and German Patent No. 40 33 563.

TOFA has poor bioavailability through the skin. On the other hand, certain prodrugs of TOFA have been found to be particularly effective against a range of dermatological disorders including acne vulgaris, acne conglobata, choracne, rosacea, Rhinophyma-type rosacea, seborrhea, seborrheic dermatitis, sebaceous gland hyperplasia, Meibomian gland dysfunction of facial rosacea, mitogenic alopecia, and oily skin. See U.S. Pat. No. 8,884,034, in the name of Dermira (Canada) Inc.

In particular, certain TOFA prodrugs can penetrate the skin and accumulate in subcutaneous hydrophobic environment such as sebaceous glands. The prodrugs then metabolize into the active TOFA form. These TOFA prodrugs are represented by the following generic formula:

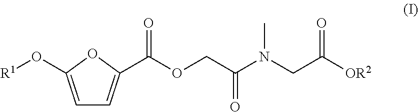

There remains a need in the art to provide dermatological formulations of TOFA prodrugs for topical applications.

### BRIEF SUMMARY

Provided herein are low-impurity drug product compositions and dermatological formulations of TOFA prodrug of Formula (I) or more specifically Formula (Ia).

One embodiment provides a drug product composition comprising a compound of Formula (I)

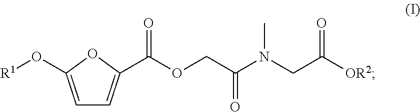

one or more impurities selected from the group consisting of:

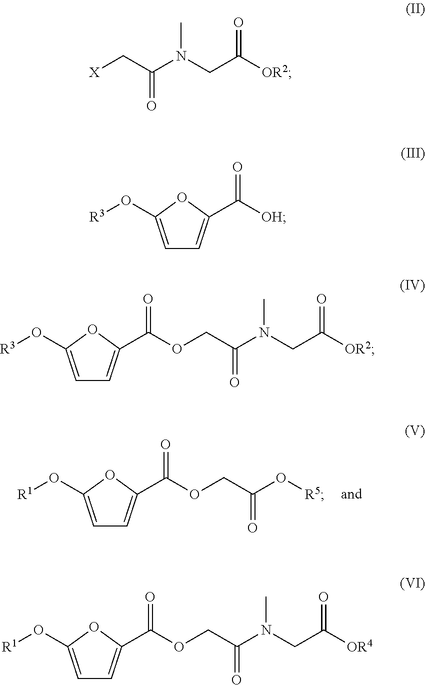

wherein,

R¹ is C₁₀₋₂₀ alkyl;

R² is C₁₋₄ alkyl;

R³ is C₁₀₋₂₀ alkyl, provided that R³ is not the same as R¹;

R⁴ is hydrogen, —(CH₂)C(O)N(CH₃)CH₂C(O)OR², or C₁₋₄ alkyl, provided that R⁴ is not the same as R²;

R⁵ is methyl or ethyl; and

X is halo,

and wherein, the one or more impurities together are no more than 3% w/w of the drug product composition.

A more specific embodiment provides a drug product composition comprising a compound of Formula (Ia)

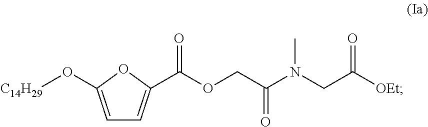

and

an impurity represented by

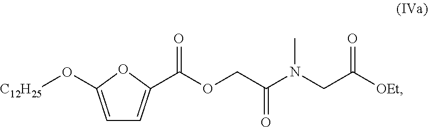

wherein, the impurity of Formula (IVa) is present at no more than 1% w/w of the drug product composition.

Another embodiment provides a dermatological formulation comprising:

a compound of Formula (I)

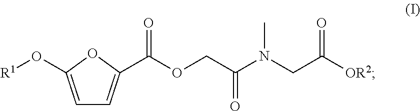

a dermatologically acceptable vehicle; and

one or more impurities selected from the group consisting of:

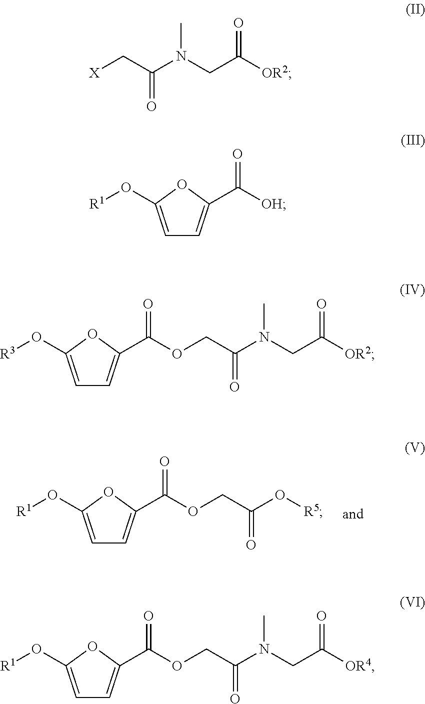

wherein,

R¹ is C₁₀₋₂₀ alkyl;

R² is C₁₋₄ alkyl;

R³ is C₁₀₋₂₀ alkyl, provided that R³ is not the same as R¹;

R⁴ is hydrogen, —(CH₂)C(O)N(CH₃)CH₂C(O)OR², or C₁₋₄ alkyl, provided that R⁴ is not the same as R²;

R⁵ is methyl or ethyl; and

X is halo,

and wherein the one or more impurities together are no more than 3% w/w of the compound of Formula (I).

A more specific embodiment provides a dermatological formulation comprising a compound of Formula (Ia):

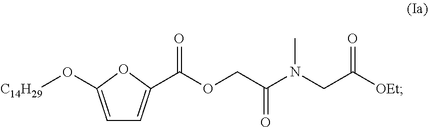

a dermatologically acceptable vehicle; and

an impurity represented by Formula (IVa):

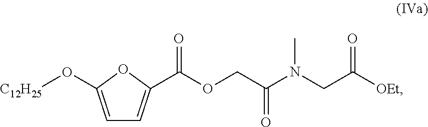

wherein, the impurity of Formula (IVa) is present at no more than 1% w/w of the compound of Formula (Ia).

A more specific embodiment provides a dermatological formulation comprising a compound of Formula (Ia) for use in a method of treating acne vulgaris, characterized in that the dermatological composition is administered topically to a subject in an area affected by acne vulgaris at least once daily, and further characterized in that the compound of Formula (Ia) is present in the dermatological formulation at a concentration of 5% (w/w) or less and an impurity represented by Formula (IVa) is present at no more than 1% w/w of the compound of Formula (Ia):

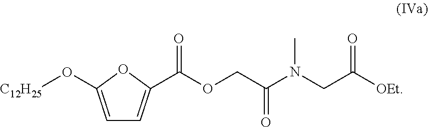

### DETAILED DESCRIPTION

Described herein include compositions comprising a TOFA prodrug of Formula (I):

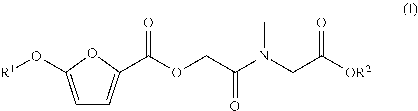

wherein R¹ is C₁₀₋₂₀ alkyl and R² is C₁₋₄ alkyl.

A more specific embodiment provides a composition comprising a compound of Formula (Ia), also named 2-((2-ethoxy-2-oxoethyl)(methyl)amino)-2-oxoethyl 5 (tetradecyloxy) furan-2-carboxylate:

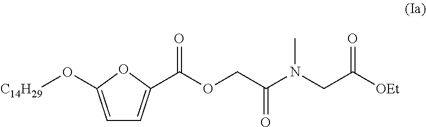

The composition may be a drug product composition (e.g., obtained from processes of pharmaceutical batch production and post-production purification) or a dermatological formulations for topical applications, in particular, at an effective amount for treating acne or reducing or inhibiting serum production. In particular, the compositions disclosed herein are characterized by a low level of impurities (not exceeding certain concentrations) to ensure safety and stability of the prodrugs of Formula (I), e.g., the compound of Formula (Ia).

### Impurities

Impurities are most likely introduced during synthetic processes that yield the active ingredients (i.e., compounds of Formula (I) or (la)). Purification of the reactants, intermediates and crude products are capable of eliminating or significantly reducing the amount of the impurities in the final product. However, to the extent that some trace amount of impurities may be present in the drug product composition or the dermatological formulation comprising the active ingredient of Formula (I), they are in amounts unlikely to cause any adverse effect to a subject or cause instability of the active ingredient during storage.

The prodrug compounds of Formula (I) are typically synthesized by coupling a compound of Formula (II) with a compound of Formula (III):

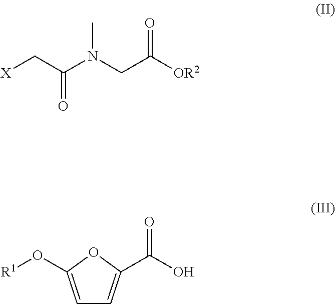

wherein, R¹ is C₁₀₋₂₀ alkyl; R² is C₁₋₄ alkyl; and X is a leaving group, as defined herein.

The first reactant, compound of Formula (II), may be synthesized by known methods in the art, including for example, the Schotten-Baumann reaction involving sarcosine ethyl ester and haloacetyl chloride (e.g., chloroacetyl chloride):

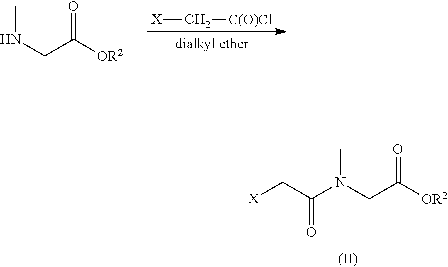

The second reactant, compound of Formula (III), e.g., TOFA, may be obtained from commercial sources or be synthesized according to the process involving an alcohol R¹—OH, as disclosed in PCT/US2016/016619. PCT/US2016/016619 is in the name of Dermira Inc., the assignee of the present application.

PCT/US2016/016616 (also in the name of Dermira Inc.) describes a synthetic process that effectively increases the yields of the compounds of Formula (I) in scaled-up batch productions, while minimizing the level of impurities in the crude product. Examples 1 and 2 describe the synthetic preparation of Formula (Ia) in more detail.

It was discovered that the impurities typically associated with a post-synthesis crude product of compound of Formula (I) may be degradants, residual reactants, or downstream by-products formed by impurities in the first reactant with the second reactant or vice versa. Thus, as used herein, an impurity may be a compound containing one or more chemical motifs of either compound of Formula (II) or compound of Formula (III). In particular, an impurity may be a structural analog of the compound of Formula (I), sharing structural motifs such as the 5-alkoxy furan-2-carboxylate esters. An impurity may also be an unreacted reactant, i.e., a compound of Formula (II) or (III).

More specifically, these impurities include one or more of the followings:

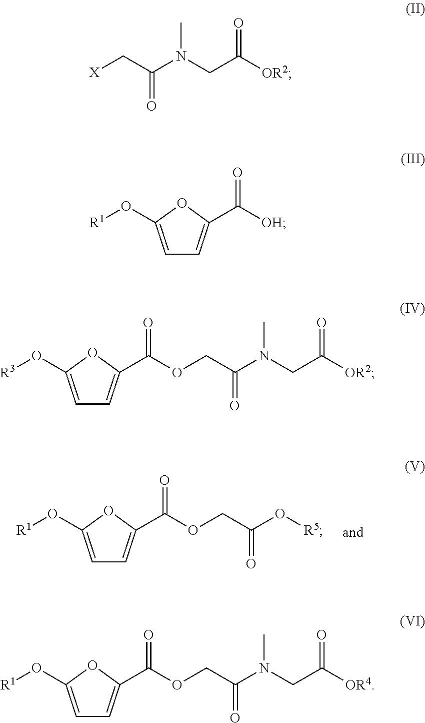

wherein,

R¹ is C₁₀₋₂₀ alkyl;

R² is C₁₋₄ alkyl;

R³ is C₁₀₋₂₀ alkyl, provided that R³ is not the same as R¹;

R⁴ is hydrogen, —(CH₂)C(O)N(CH₃)CH₂C(O)OR², or C₁₋₄ alkyl, provided that R⁴ is not the same as R²;

R⁵ is methyl or ethyl; and

X is halo.

In more specific embodiments, wherein the compound of Formula (I) is represented by Formula (Ia), the impurities may be one or more of the following:

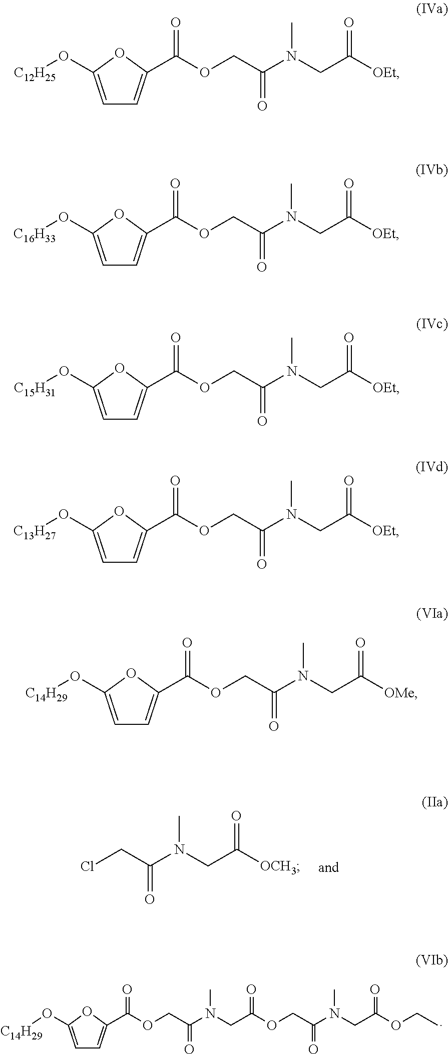

As disclosed herein, the synthesis process may be refined by using purer reactants and the crude product can be further purified to remove certain specific impurities such as residual reactants. As a result of the improved synthesis and post-production purification steps, such as those disclosed PCT/US2016/016616 and PCT/US2016/016619, the total amount of the one or more impurities, including but not limited to compounds represented by any one of Formulae (II)-(VI) or substructures thereof, does not exceed 3% w/w of a given composition (e.g., a drug product composition). In more preferred embodiments, the total amount of the one or more impurities does not exceed 2% w/w of a composition or does not exceed 1% w/w of a drug product composition. In certain embodiments, none of the impurities represented by Formulae (II)-(VI), including for example, substructures represented by Formulae (IIa), (IVa-IVd), and (VIa-VIb), are present in a drug product composition.

### Drug Product Compositions

As used herein, a drug product or a drug product composition refers to a composition comprising a prodrug of Formula (I), or specifically Formula (Ia), as the active ingredient (“drug”). The drug product composition may be batch products from commercial manufacturing facilities, including GMP facilities. In certain embodiments, the drug product may contain no impurity (i.e., 100% active ingredient). In other embodiments, the drug product contains one or more impurities of Formulae (II)-(VI), the total amount of which does not exceed 3% w/w of the total weight of the drug product composition.

In other embodiments, the total amount of the one or more impurities of Formulae (II)-(VI) does not exceed 2% w/w of the total weight of the drug product composition. In preferred embodiments, the total amount of the one or more impurities of Formulae (II)-(VI) does not exceed 1% w/w of the total weight of the drug product composition.

In certain embodiments, the active ingredient of Formula (I) in the drug product is at least 97% w/w of the total weight of the drug product. In preferred embodiments, the active ingredient of Formula (I) in the drug product is at least 98% or at least 99% w/w of the total weight of the drug product.

In one embodiment, the drug product composition is a GMP batch product having at least 3 kg of the compound of Formula (I). In another embodiment, the drug product composition is a GMP batch product having at least 40 kg of the compound of Formula (I). In further embodiment, the drug product composition is a GMP batch product having at least 100 kg of the compound of Formula (I) (e.g., a compound of Formula (Ia)).

In certain embodiments, the impurity represented by Formula (IV) is no more than 2% w/w of the composition. Impurities of Formula (IV) are likely downstream by-products derived from alcohol impurities in the reactant R¹—OH, such as trace amount of R³—OH (wherein R³ is different from R¹). In various embodiments, when R¹ is —C₁₄H₂₉, R³ may be —C₁₂H₂₅, —C₁₃H₂₇, —C₁₅H₃₁, —C₁₆H₃₃, or —C₁₈H₃₇ alkyl.

In certain embodiments, the impurity represented by Formula (II) is no more than 0.5% of the composition; more preferably, no more than 120 ppm of the composition. of the drug product composition. Formula (II) is a reactant of the coupling reaction to produce the compound of Formula (I). Compounds of Formula (II) have an α-halocarbonyl structural motif that may be toxic. In certain embodiments, X (the halo group of the halocarbonyl) is bromo or chloro. Thus, to the extent that a trace amount of unreacted Formula (II) may remain in the drug product composition, the amount does not exceed 0.5%, or preferably does not exceed 120 ppm of the composition.

In certain embodiments, impurities represented by Formula (VI) are no more than 0.5% w/w of the composition. These impurities include degradants of the active ingredient, i.e., the prodrug compound of Formula (I). In certain specific embodiments, R⁴ is hydrogen or methyl.

In other embodiments, impurities represented by Formula (V) are no more than 0.2% w/w of the composition.

In specific embodiments, the active ingredient of a drug product composition, i.e., the compound of Formula (I) is represented by Formula (Ia):

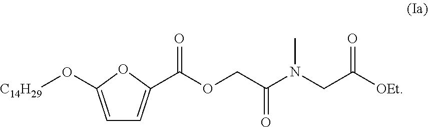

The impurities typically associated with manufacturing the compound of Formula (Ia) include one or more of the following compounds:

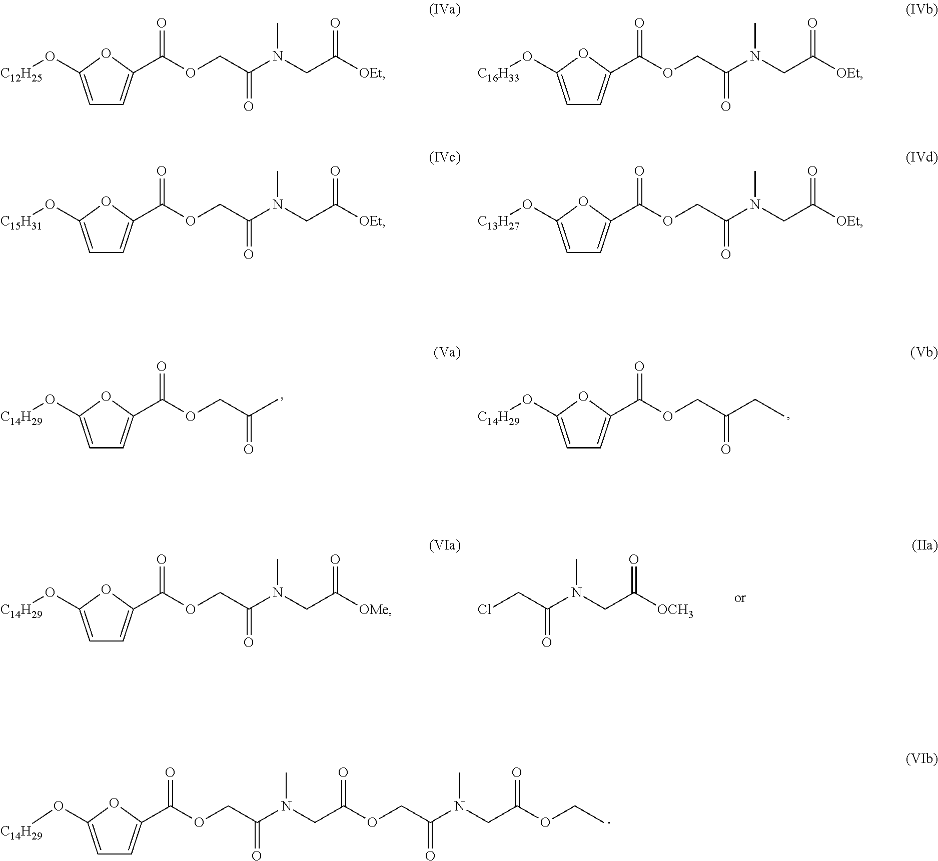

A specific embodiment thus provides a drug product composition comprising a compound of Formula (Ia)

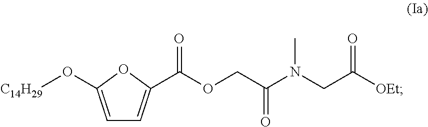

an impurity represented by Formula (IVa):

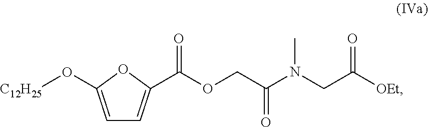

wherein, the compound of Formula (Ia) is at least 97% w/w of the drug product composition and the impurity of Formula (IVa) is present at no more than 1% w/w of the drug product composition.

In a further more specific embodiment, the drug product composition comprises an impurity represented by Formula (IVb):

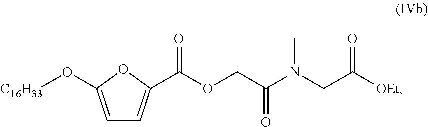

wherein, the impurity of Formula (IVb) is present at no more than 0.5% w/w of the drug product composition.

In a further more specific embodiment, the drug product composition further comprising an impurity represented by Formula (VIa):

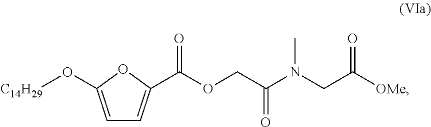

wherein, the impurity of Formula (VIa) is present at no more than 0.3% w/w of the drug product composition.

In a further more specific embodiment, the drug product composition further comprising an impurity represented by Formula (VIb):

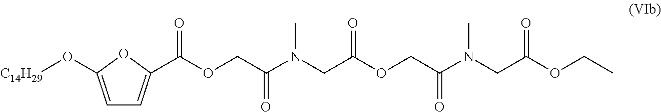

wherein, the impurity of Formula (VIb) is present at no more than 0.5% w/w of the drug product composition, or no more than 0.3% w/w of the drug product composition.

In yet further more specific embodiment, the drug product composition further comprising an impurity represented by Formula (IIa):

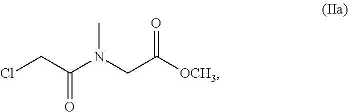

wherein, the impurity of Formula (IIa) is present at no more than 120 ppm w/w of the drug product composition.

In other embodiments, the total amount of the one or more impurities of Formulae (IVa), (IVb), (Va), (Vb), (VIa), (VIb) and (IIa) does not exceed 2% w/w of the total weight of the drug product composition. In preferred embodiments, the one or more impurities of Formulae (IVa), (IVb), (Va), (Vb), (VIa), (VIb) and (IIa) does not exceed 1% w/w of the total weight of the drug product composition.

In certain embodiments, the active ingredient of Formula (Ia) in the drug product is at least 97% w/w of the total weight of the drug product. In preferred embodiments, the active ingredient of Formula (Ia) in the drug product is at least 98% or at least 99% w/w of the total weight of the drug product.

### Dermatological Formulations

The drug product disclosed herein may be further formulated into dermatological formulations for topical uses. Depending on the strength, the concentrations of the active ingredient, i.e., Formula (I) or specifically Formula (Ia), may vary. In various embodiments, the active ingredient has a concentration of 1-10% w/w (excluding 1%) of the total weight of the dermatological formulation. In certain embodiments, the compound of Formula (Ia) is present in the dermatological formulation at a concentration (w/w) of more than 1%, but no more than 7.5%, or no more than 7%, or no more than 6%, or no more than 5%, or no more than 4%, or no more than 3%. In preferred embodiments, the compound of Formula (Ia) has a concentration of 2%, 4%, 5%, 6%, 7% and 7.5% w/w of the total weight of the dermatological formulation.

The major component of the dermatological formulations disclosed herein is a dermatologically acceptable vehicle, in which the active ingredient is dissolved or suspended. The dermatologically acceptable vehicle may contain one or more agents such as adjuvant, carrier, excipient, glidant, diluent, preservative, fragrance, dye/colorant, surfactant, wetting agent, dispersing agent, suspending agent, thickening agent, skin-penetration enhancer, stabilizer, isotonic agent, solvent, or emulsifier, including those approved by the United States Food and Drug Administration as being acceptable for dermatological use on humans or domestic animals, or which are known, or are suitable for use in dermatological formulations.

The nature and composition of the dermatologically acceptable vehicle determine the form (e.g., cream, gel, solution, lotion, foam, ointment, etc.) of the dermatological formulation. In a specific embodiment, the dermatological formulation is an alcohol-based gel. Because the compounds of Formula (I) tend to have poor solubility in water, in certain specific embodiments, the alcohol-based gel is non-aqueous.

In various embodiments, the dermatologically acceptable vehicle comprises dimethyl isosorbide and one or more alcohols. Dimethyl isosorbide (DMI) is a solvent in which a compound of Formula (I), specifically Formula (Ia), has high solubility (about 125 mg/g). DMI is freely miscible with alcohols such as ethanol, isopropanol (IPA), polyols such as polyethylene glycol (PEG 200 or PEG 400), or a mixture thereof. By adjusting the relevant amounts of DMI and the alcohol(s), the solubility and saturation of the active ingredient in the dermatological formulation can be adjusted to maximize the thermodynamic activity of the active ingredient in the gel. In a specific embodiment, the dermatologically acceptable vehicle comprises, by weight ratios, 50 parts ethanol, 20 parts IPA, 15.5 parts PEG400, and 12.5 parts DMI. The vehicle is typically a clear gel, and bears the same appearance with or without the active ingredient.

Formulations with lower alcohol contents may be desirable for skin types prone to irritation or dryness. In lower alcohol-content formulations, a skin penetration enhancer may be optionally added to ensure delivery of the active ingredient through the skin. An example of skin penetration enhancer is diethylene glycol monoethyl ether (Transcutol® P). Table 1 shows four exemplary formulations:

TABLE 1

| | Formula- | Formula- | Formula- | Formula- |
|---|---|---|---|---|
| | tion 1 | tion 2 | tion 3 | tion 4 |
| Arlasolve DMI | 11.54 | — | — | — |
| Ethanol | 46.18 | 48.34 | 38.67 | 33.57 |
| IPA | 18.47 | 19.34 | 9.67 | 4.80 |
| PEG-400 | 14.31 | 27.07 | 46.41 | 31.65 |
| Transcutol P | | | | 23.98 |
| HPC | 2.00 | 2.00 | 2.00 | 2.00 |
| Formula (la) | 7.50 | 3.25 | 3.25 | 4.00 |
| Strength (mg/g) | 7.5% | 3.25% | 3.25% | 4.00% |

The amount of any impurities (e.g., compounds of Formulae (II)-(VI) and their substructures) present the in dermatological formulations, are measured against the amount of the active ingredient by % w/w. Thus, certain embodiments provide a dermatological formulation comprising: a compound of Formula (I)

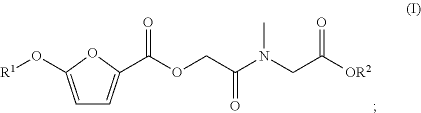

a dermatologically acceptable vehicle; and

one or more impurities selected from the group consisting of:

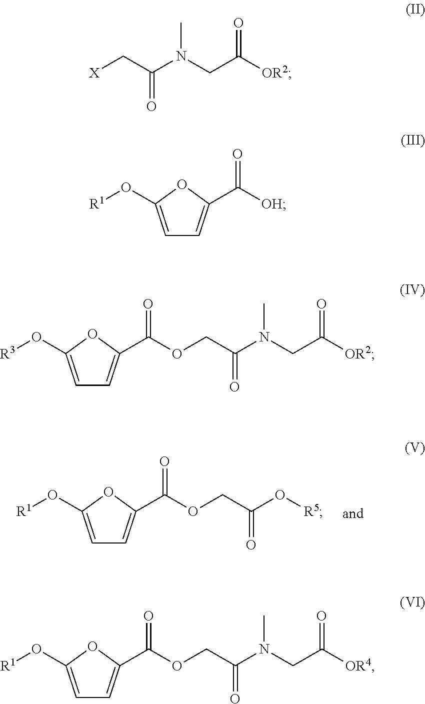

wherein,

R¹ is C₁₀₋₂₀ alkyl;

R² is C₁₋₄ alkyl;

R³ is C₁₀₋₂₀ alkyl, provided that R³ is not the same as R¹;

R⁴ is hydrogen, —(CH₂)C(O)N(CH₃)CH₂C(O)OR², or C₁₋₄ alkyl, provided that R⁴ is not the same as R²;

R⁵ is methyl or ethyl; and

X is halo, and wherein the one or more impurities together are no more than 3% w/w of the compound of Formula (I).

In other embodiments, the one or more impurities represented by Formula (IV) are no more than 2% w/w, or no more than 1.5%, or no more than 1% or no more than 0.5% w/w of the compound of Formula (I).

In further embodiments, one or more impurities represented by Formula (II) are no more than 120 ppm of the compound of Formula (I).

In yet further embodiments, the byproduct represented by Formula (VI), wherein R⁴ is hydrogen, is no more than 0.5% w/w of the compound of Formula (I).

In yet further embodiments, the byproduct represented by Formula (VI), wherein R⁴ is —(CH₂)C(O)N(CH₃)CH₂C(O)OR², is no more than 0.5% w/w of the compound of Formula (I), or no more than 0.3% w/w of the compound of Formula (I).

In further embodiments, the one or more impurities represented by Formula (V) are no more than 0.2% w/w of the compound of Formula (I).

A more specific embodiment provides a dermatological formulation comprising: a compound of Formula (Ia):

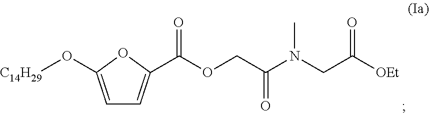

a dermatologically acceptable vehicle; and

an impurity represented by Formula (IVa):

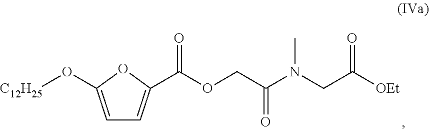

wherein, the impurity of Formula (IVa) is present at no more than 1% w/w of the compound of Formula (Ia). In further more specific embodiments, the impurity of Formula (Iva) is present at no more than 0.5% or no more than 0.1% w/w of the compound of Formula (Ia).

In another embodiment, the dermatological formulation further comprises an impurity represented by Formula (IVb):

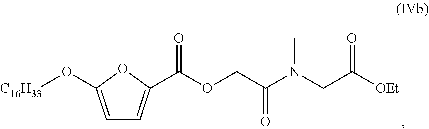

wherein, the impurity of Formula (IVb) is present at no more than 0.5% w/w of the compound of Formula (Ia). In further more specific embodiments, the impurity of Formula (IVb) is present at no more than 0.1%, or no more than 0.05% w/w of the compound of Formula (Ia).

In various embodiments, the dermatological formulation further comprises an impurity represented by Formula (VIa):

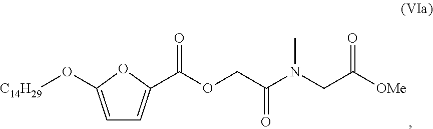

wherein, the impurity of Formula (Via) is present at no more than 0.3% w/w of the compound of Formula (Ia). In further more specific embodiments, the impurity of Formula (IVa) is present at no more than 0.1%, or no more than 0.05% w/w of the compound of Formula (Ia).

In yet further embodiments, the by-product is represented by Formula (VIb):

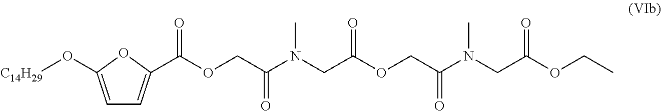

wherein the impurity of Formula (VIb) is no more than 0.5% w/w of the compound of Formula (Ia), or no more than 0.3% w/w of the compound of Formula (Ia), or no more than 0.1% w/w of the compound of Formula (Ia).

In other various embodiments, the dermatological formulation further comprises an impurity represented by Formula (IIa):

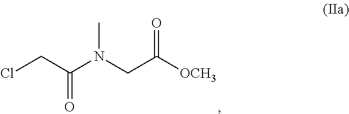

wherein, the impurity of Formula (IIa) is present at no more than 0.5% or no more than 120 ppm of the compound of Formula (Ia).

### Purification and Stability of Compound of Formula (Ia)

The compound of Formula (Ia) may be prepared and purified into a crystalline product form. More specifically, the product may be purified by recrystallization in an alcoholic solvent including, for example, isopropanol.

In certain embodiment, the crystalline product form is a white crystalline solid with a low-melting point (64-66° C.), having solubility of about 90 mg/g (±5 mg/g) in a solvent system comprising Ethanol/IPA/PEG 400/DMI at 50/20/15.5/12.5 (w/w).

Stability studies conducted on non-GMP and GMP material have shown chemical stability the compound of Formula (Ia) for 24 months at 5° C. and 25° C./60% Relative Humidity (RH); and for 6 months at 40° C./75% RH. The stability of the crystal form has also been demonstrated at 5° C. and 25° C./60% RH conditions for up to 36 months and at 30° C./65% RH for up to 27 months.

In some embodiments, the compound of Formula (Ia) is present in a formulation at a concentration at or below which the formulation remains stable for an extended period of time (e.g., 24 months or longer) without degradation or precipitation. In various specific embodiments, the compound of Formula (Ia) is at a concentration of 7.5% or less, or 7% or less, or 6% or less, or 5% or less, or 4% or less, or 3% or less, or 2% or less (w/w) in a dermatological formulation. Preferably, the compound of Formula (Ia) has a concentration (or strength) of 5% or less in a dermatological formulation.

### Method of Treatment and Pharmaceutical Use

Acne or acne vulgaris is a common skin disease characterized by clogging of the pores and associated local skin lesions that usually appear on the face, chest or back. Acne lesions are believed to result from an interaction of four primary pathogenic factors, including (1) excessive production of sebum by sebaceous glands or sebaceous gland hyperactivity; (2) alterations in skin cells that contributes to clogging of pores through which sebum is normally released to the skin surface; (3) colonization of the sebaceous gland by bacteria that are nourished by sebum; and (4) inflammation often associated with colonization by bacteria and their digestion of sebum into breakdown products that are known to cause inflammation. Clogged pores can become enlarged and inflamed as sebum and its breakdown products accumulate, resulting in visible lesions that can be unsightly and cause permanent scarring.

Dermatological formulations comprising a compound of Formula (I), in particular, a compound of Formula (Ia), are effective topical therapy for treating acne by targeting one or more of the above factors. Advantageously, the dermatological formulations are capable of delivering an effective amount of TOFA prodrug through the skin. The prodrug subsequently converts to TOFA, which is a potent inhibitor of lipid synthesis. Thus, the prodrug compounds of Formula (I) or (la) make it possible to effectively reduce or inhibit sebaceous serum production by delivering TOFA to the sebaceous gland. TOFA (the active form of the prodrug) accumulates in the sebaceous gland and reaches a therapeutic level.

One embodiment provides a method of treating acne vulgaris or other dermatological disorder associated with sebaceous gland hyperactivity comprising administering to a subject in need thereof a dermatological formulation comprising a compound of Formula (I), e.g., Formula (Ia), wherein the compound of Formula (I) or (la) is present at a concentration of 7.5% (w/w) or less.

In other embodiments, the concentration or strength of the active ingredient is 7% or less, 6% or less, 5% or less, 4% or less or 3% or less (w/w). In certain above embodiments, the concentrations of the active ingredients are above 1% (w/w).

In various embodiments, the dermatological formulations have low or no impurities as represented by Formula (II)-(VI) and any of the substructures thereof.

In more specifically embodiments, administering the dermatological formulation comprises applying it directly and locally to the affected skin of the subject.

As used herein, affected skin refers to skin that presents at least one inflammatory or non-inflammatory lesion. The affected skin may be facial skin, or skin on the chest or back area.

In various embodiments, the dermatological formulation is administered once a day (QD), or twice a day (BID).

A specific embodiment provides a method of treating acne vulgaris comprising administering to a subject in need thereof twice daily a dermatological formulation comprising a compound of Formula (Ia), wherein the compound of Formula (Ia) is present at a concentration of 5% (w/w). In more specific embodiment, the dermatological formulation comprises an impurity represented by Formula (IVa) in an amount of no more than 1% w/w of the compound of Formula (Ia).

A further specific embodiment provides a method of treating acne vulgaris comprising administering to a subject in need thereof once daily a dermatological formulation comprising a compound of Formula (Ia), wherein the compound of Formula (Ia) is present at a concentration of 5% (w/w).

A specific embodiment provides a method of treating acne vulgaris comprising administering to a subject in need thereof twice daily a dermatological formulation comprising a compound of Formula (Ia), wherein the compound of Formula (Ia) is present at a concentration of 7.5% (w/w).

A specific embodiment provides a method of treating acne vulgaris comprising administering to a subject in need thereof once daily a dermatological formulation comprising a compound of Formula (Ia), wherein the compound of Formula (Ia) is present at a concentration of 7.5% (w/w).

The efficacy of the dermatological use of the compound of Formula (I), particularly Formula (Ia) may be assessed through lesion counts (inflammatory and non-inflammatory), investigator global assessment (IGA), sebum excretion rates (SERs), and biomarkers associated with sebum excretion according to known methods in the art. Example 3 provides more detailed description of disease severity assessment and efficacy endpoints.

Safety assessment may be carried out by observing local skin responses determined by the presence and severity of erythema, dryness, peeling, burning/stinging, and pruritus. Advantageously, there is little or no systemic absorption of Formula (Ia) or TOFA following topical application of the same at 7.5% strength, twice daily for 12 weeks.

The duration of the treatment may vary depending on the severity of acne and the local skin response under treatment. In various embodiments, the method comprises administering the dermatological formulation described herein, either once daily or twice daily, for up to 2 weeks, 4 week, 8 weeks or 12 weeks. Longer durations are possible if local skin response demonstrates tolerance.

### Combination Therapy

The dermatological formulations described herein or the treatment regimen may be combined with other topical or oral products for patients with moderate to severe acne. The combination therapy may advantageously target multiple acne pathology factors.

In various embodiments, the additional agents may include topical retinoids, topical benzoyl peroxide (BPO), topical and oral antimicrobials, topical combination products such as retinoid/antibiotic (e.g., Ziana, Veltin) and retinoid/BPO (Epiduo/Epiduo Forte), oral isotretinoin and oral hormone therapies, including sex hormones such as androgens.

Topical agents may be combined with the dermatological formulation described herein and co-administered, or administered separately (e.g., each administered once daily at different times of the day).

Thus, a specific embodiment provides administering (1) a dermatological formulation described herein; and (2) an additional topical agent selected from a retinoid, an antibiotic and benzoyl peroxide. Specific retinoids for topical use may include, for example, tretinoin (all-trans retinoic acid), polyaromatics adapalene, tazarotene, isotretinoin (13-cis retinoic acid), and adapalene and the like.

Thus, a specific embodiment provides administering (1) a dermatological formulation described herein; and (2) an additional oral agent selected from an oral antibiotic, oral isotretinoin and oral hormone therapeutic agent.

### Additional Definitions

As used herein, “alkyl” refers to a straight or branched hydrocarbon chain radical consisting solely of carbon and hydrogen atoms, containing no unsaturation, having from one to twenty four carbon atoms (C₁₋₂₄ alkyl). Long-chain alkyls include, for example, ten to twenty carbon atoms (C₁₀₋₂₀ alkyl), or ten to fifteen carbon atoms (C₁₀₋₁₅ alkyl). Alkyls may be represented by —CₘH₂ₘ₊₁ (m denotes the number of carbons). Short-chain alkyls include, for example, one to eight carbon atoms (C₁₋₈ alkyl), or one to six carbon atoms (C₁₋₆ alkyl), or one to four carbon atoms (C₁₋₄ alkyl). The alkyl radical is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group may be unsubstituted or substituted by halo (F, Cl, Br, or I), haloalkyl (e.g., CF₃), alkoxy (i.e., —O-alkyl), hydroxy (—OH), acyl group (—OC(O)alkyl) or carboxyl group.

“Leaving group” refers to a molecular fragment that is capable of being displaced (e.g., in a SN2 reaction) by a nucleophile. For example, a leaving group may be a halogen (i.e., Br, Cl or I), or a tosyl group (e.g., —OTs).

“Halo” refers to fluoro, bromo, chloro or iodo.

### EXAMPLES

### Example 1

### Preparation of Formula (Ia)

Compound of Formula (Ia), 2-(2-ethoxy-2-oxoethyl)(methyl)amino-2-oxoethyl 5-(tetradecyloxy)furan-2-carboxylate (shown as 4), was prepared according to the following general reaction Scheme 1:

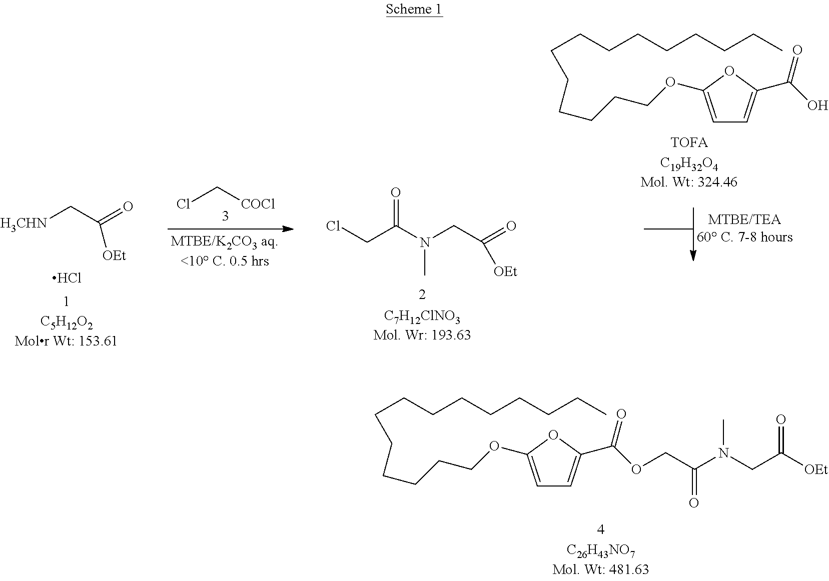

### Step 1: Preparation of Side Chain Reactant (2)

Compound (2), which forms the ester side chain of TOFA in the compound of Formula (Ia), was prepared by acylation of Compound (1) under Schotten-Baumann conditions. More specifically, an aqueous solution of potassium carbonate and chloroacetyl chloride (3) was added to a vigorously stirred suspension of sarcosine ethyl ester hydrochloride (1) in a dialkyl ether (e.g., methyl t-butyl ether, or “MTBE”). The reaction proceeded quantitatively at ambient temperature within about 30 minutes. The crude reaction mixture can be optionally diluted with the dialkyl ether solvent (MTBE), and underwent phase separation. After the aqueous phase was removed, the title compound (2), which was present in the organic layer (i.e. MTBE), could be used directly for the coupling step (Step 3).

The Schotten-Baumann conditions could also be slightly modified to produce compound (2) as follows. To a mixture of 0.307 g (2.0 mmol) of sarcosine ethyl ester hydrochloride (1) in EtOAc (3 mL) and 3 mL of saturated NaHCO₃ solution was added chloroacetyl chloride (3) (0.160 mL, 2 mmol). Effervescence was observed. Once gas production had ceased, the reaction mixture was diluted with ethyl acetate (10 mL). The phases were separated and the organic phase was washed with brine (5 mL), dried and concentrated to yield ˜0.250 g of the title compound (2) as an oil. The crude material was used in the subsequent step without further purification.

The above processes were shown to be scalable with minor changes. An output scale of 13 kg (corrected for purity) with yields varying from 60-80% could be consistently obtained.

### Step 2—Scale-Up Synthesis of TOFA

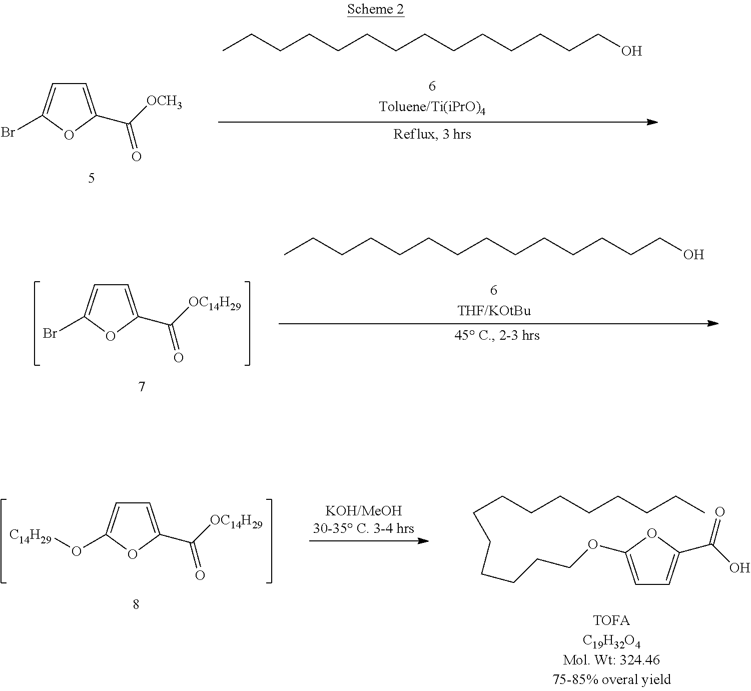

TOFA was prepared according to the above synthetic route. More specifically, methyl ester of 5-bromo-2-furoic acid (5) first underwent transesterification with 1-tetradecanol (6) (about 1 eq) in the presence of titanium tetraisopropoxide in refluxing toluene with removal of the methanol formed to provide tetradecyl ester of 5-bromofuroic acid (7). Thereafter, THF was added, and the transesterification product (7) was treated with tetradecoxide (i.e., potassium salt of tetradecanol 6), which was prepared by combining potassium t-butoxide or potassium t-pentoxide with tetradecanol.

The reaction was carried out rapidly at a low temperature of 45° C. to produce mixed esters of TOFA, including predominately tetradecyl ester of TOFA (8) and about 5-10% t-butyl ester of TOFA (structure not shown). Other by-products such as methyl ester of TOFA might also be present in small amounts.

Thereafter, the mixed esters were saponified by treatment with methanolic KOH for 3-4 hours at low temperature of 30-35° C. to produce TOFA in about 75-85% overall yield.

### Step 3—Coupling of (2) and TOFA

The coupling reaction was conducted over 7-8 hours in MTBE under reflux (˜60° C.) in the presence of a suitable base such as triethylamine (TEA). After aqueous work-up using a phosphate buffer, the organic phase underwent solvent exchange to 2-propanol. Crystallization of the coupling product (4) was induced by addition of water. The crystalline product was isolated at about 83% yield from TOFA. Advantageously, because the same solvent (MTBE) could be used in both Steps 1 and 3, the top volume of the claimed process could be less than half that of the conventional process, thereby significantly improving throughput.

By using higher purity tetradecanol (6), downstream impurities such as Formula (IVa) and (IVb) were effectively reduced. In addition, using an isopropanol trituration of the crude prodrug (4), i.e., Formula (Ia), side chain intermediate and residual tetradecanol were further removed. The purity level of the prodrug was able to reach at least 97%, or at least 98% or at least 99% of the drug product composition.

### Example 2

### Batch Production of Formula (Ia)

In large-scale pharmaceutical batch productions, care was taken to minimize production of by-products by monitoring completion of reaction or to purify reaction intermediates at various stages of the synthetic processes. As disclosed in this Example, identification and elimination of the byproducts from reaction intermediates resulted in significantly reduced and controllable amounts of impurities in the final pharmaceutical product.

### Manufacture of 5-(Tetradecyloxy)-2-furoic acid (TOFA)

In accordance with the general reaction Scheme 2, methyl-5-bromo-2-furoate (5) (110 kg; 0.536 kmol), 1-tetradecanol (6) (253 kg; 1.18 kmol), and toluene (900 L), titanium tetraisopropoxide (3.85 kg; 0.0135 kmol) were charged in a 4000 L reactor, which had been previously rinsed with toluene (200 L). The reaction mixture was heated to reflux (approximately 115-135° C.) with agitation for at least 4 h. The total volume was reduced to approximately one-third of the original volume using atmospheric distillation. The reaction mixture was cooled to approximately 30° C. and sampled for analysis. The mixture was analyzed by UPLC to confirm that the level of residual methyl-5-bromo-2-furoate (5) with respect to reaction intermediate (7) is no more than 2%. In addition, the methanol content was 50.1% w/w with respect to toluene by GC analysis. Additional distillation cycles may be performed until acceptance criteria are met.

Once the acceptance criterion was met, THF (1120 L) was added and the reaction mixture was then heated to approximately 40-45° C. A solution of 20% potassium tert-butoxide in THF (354.5 kg; 0.64 kmol) was added over approximately one hour, while maintaining the temperature below 55° C. The mixture obtained was stirred at approximately 50-55° C. for about 2-3 hours, at which point it was sampled and analyzed by UPLC for reaction completion. The reaction intermediate, tetradecyl ester of TOFA (8), was accompanied by minor amounts of methyl ester of TOFA (9) and t-butyl ester of TOFA (10). The reaction is considered complete when the ratio of the sum of (5) and (7) to the sum of the TOFA esters (including 8, 9 and 10), i.e., Σ(5+7):Σ(8+9+10), is ≦1% a/a. The mixture of the TOFA esters (8, 9 and 10) was not isolated before undergoing the next saponification step. Instead, the mixture was directly treated with a solution of potassium hydroxide in methanol (60.5 kg in 297 L). The resulting mixture was agitated for about 4 hours at approximately 40-45° C. before being sampled and analyzed by UPLC for reaction completion. The reaction is considered complete when the ratio of the sum of the TOFA esters to TOFA, i.e., Σ(8+9+10):TOFA, is ≦0.5% a/a.

### Work-Up and Purification of TOFA

The above reaction mixture was first neutralized and the pH further adjusted to approximately 3.5-4.0 with 20% aqueous phosphoric acid (732 kg). The lower aqueous layer was drained and the organic phase was maintained at approximately 40-45° C. While maintaining the temperature at approximately 40-45° C., xylenes (759 kg) were added followed by water (550 L). The mixture was agitated for about 30 minutes and the lower aqueous layer drained. The volume of the organic layer was reduced to approximately half under vacuum. The mixture was then sampled and analyzed by GC to confirm that Σ(MeOH+THF+toluene):xylenes is ≦5%. If the solvent ratio is not achieved, xylenes (704 kg) should be added and distillation cycles should continue until the acceptance criterion is met.

The solution was allowed to cool to approximately 23° C. to crystallize the product. The mixture was stirred for a minimum 2 hours and the product recovered by filtration. The cake formed after filtration was washed with xylenes (187 kg) then n-heptane (220 L), and finally dried on the filter under vacuum under a nitrogen stream, under 40° C., until the loss on drying is ≦2%. If the tetradecanol level is >2% the product may be slurried in approximately 5 volumes of xylenes for 5 h, filtered, washed with n-heptane and dried under a nitrogen stream until loss on drying is ≦2%. The yield of TOFA was typically 132.4 kg (76%).

### Manufacture of Side Chain Reactant (2)

In accordance with general reaction Scheme 1, sarcosine ethyl ester hydrochloride (1) (103.4 kg; 0.673 kmol) and MTBE (671 L) were charged to a reactor, followed by an aqueous solution of potassium carbonate (190.3 kg; 1.38 kmol in 539 L water), while maintaining a temperature of below 10° C. The mixture was cooled to approximately 0-5° C., and chloroacetyl chloride (3) (91.9 kg; 0.81 kmol) was added at such a rate to maintain the temperature below 15° C. The reaction mixture was warmed to approximately 20-25° C., and the lower aqueous layer was removed and the organic layer, which contained the side chain reactant (2), was washed with monobasic potassium phosphate solution (30.8 kg; pH 3.0-4.0). To the solution of (2) was added MTBE (550 L) and then concentrated by atmospheric pressure distillation to about half of the original volume, using a jacket temperature of 75° C. The moisture content of the solution was determined by Karl Fisher titration. Additional MTBE is added and the distillation is repeated until the moisture content of the solution is 50.3%. The solution of (2) was assayed for content used as such in the coupling reaction. An assay of (2) was obtained to ensure that the amount of (2) is >1.3 equiv. with respect to the amount of TOFA to be used. If not, the amount of TOFA used in the coupling reaction is adjusted so that the molar ratio of (2):TOFA>1.3.

### Manufacture of the Compound of Formula (Ia)

In accordance with general reaction Scheme 1, the product (4), i.e., the compound of Formula (Ia), was prepared in large-scale. A reactor was charged with TOFA (110 kg; 0.34 kmol) followed by the solution of the side chain reactant (2) (1.4 equiv), followed by triethylamine (68.2 kg; 0.68 kmol). The reaction mixture was heated at reflux for a minimum of 5 h. The reaction mixture was cooled to about 40-50° C. and analyzed by HPLC to monitor the completion of reaction (ratio of the remaining TOFA to product 4 is ≦0.2%). The reaction was heated at reflux until the in-process control criterion is achieved. The reaction mixture was cooled to 20-25° C., diluted with MTBE (220 L) and acidified with approximately 1.3 equiv. of 1M KH₂PO₄ buffer at a pH of 3.0-4.0 (773.4 kg). The lower aqueous layer was removed and the organic layer was washed three times with 1% monobasic phosphate buffer (550 L) and polish filtered into a clean reactor. The reactor was rinsed with MTBE (550 L) and the MTBE solution of the product (4) was concentrated to approximately 6 vol of solvent. The mixture was cooled to approximately 40° C. and heptane (682 L) was added, cooled to approximately 30° C. and seeded with 220 g of crystalline compound of Formula (Ia), which had been previously purified and recrystallized. After stirring for an hour at 30° C. the mixture was cooled to 10° C. over about 2 h, and aged for 10 h at that temperature. The crude product was filtered and washed with 1:1 MTBE/heptane (220 L).

The crude wet product (318 kg) was dissolved in MTBE (770 L) by heating to about 45° C., and then polish filtered into a clean reactor and rinsed forward with MTBE (110 L). The MTBE solution at about 45° C. was treated with heptane (770 L) and cooled to about 30° C., and seeded with 220 g of crystalline form of the compound of Formula (Ia). The solution was maintained at 30° C. for about 1 h, then cooled to 18° C. over the period of about 1 h, and maintained at that temperature for 3-4 h. The slurry was heated to 30° C. over the period of about 1 h and maintained at that temperature for about 20 h. The slurry was cooled to 18° C. over 1 h and maintained at 18° C. for an additional hour. The product was isolated by centrifugation, washed with 1:1 MTBE/heptane (330 L) and dried under vacuum oven at no more than 40° C. The overall yield of purified product (4), based on TOFA, was 132 kg (81%).

### Example 3

### Global Assessment of Disease Severity and Efficacy Endpoints

Disease severity was scored using a 5-point Investigator Global Assessment (IGA) for acne (see Table 2).

TABLE 2
Investigator Global Assessment (IGA)

| Grade | Description |
|---|---|
| 0 | Clear; normal, clear skin with no evidence of acne vulgaris |
| 1 | Almost clear; Rare non-inflammatory lesions present, with |
| | rare non-inflamed papules (papules must be resolving and |
| | may be hyperpigmented, though not pink-red) |
| 2 | Mild; some non-inflammatory lesions are present, with few |
| | inflammatory lesions (papules/pustules only; no |
| | nodulocystic lesions) |
| 3 | Moderate; non-inflammatory lesions predominate, with |
| | multiple inflammatory lesions evident: several to many |
| | comedones and papules/pustules, and there may or may |
| | not be one small nodulocystic lesions |
| 4 | Severe; Inflammatory lesions are more apparent, many |
| | comedones and papules/pustules, there may or may not |
| | be a few nodulocystic lesions |

### Example 4

### Efficacy of Topical Treatment by Formula (Ia)

Approximately 100 subjects having acne vulgaris were randomized in a 1:1 ratio to active formulation (7.5% of compound of Formula (Ia) as a topical gel formulation) or vehicle formulations (also as a topical gel). Subjects were instructed to apply formulations to the face twice daily for 12 weeks. Subjects were contacted or be assessed by clinicians at Weeks 1, 2, 3, 4, 8, 12, and 16 (study exit).

The primary efficacy endpoints were based on the: 1) absolute change from baseline at Week 12 in inflammatory and non-inflammatory acne lesion counts, and 2) proportion of subjects achieving at least a 2-pt drop in the IGA score compared to baseline at Week 12. See scoring criteria in Table 2.

Subjects treated for 12 weeks with the active formulation had significantly greater decreases from baseline in both inflammatory and non-inflammatory lesion counts than subjects treated with the vehicle gel. LS mean±SE changes in inflammatory lesion counts were −19.9±1.1 for subjects in the active formulation group and −14.3±1.1 for subjects in the vehicle formulation group (p=0.0003). LS mean±SE changes in non-inflammatory lesion counts were −20.1±1.9 for subjects in the active formulation group and −12.4±1.9 for subjects in the vehicle formulation group (p=0.0032).

For the ITT population, significantly more subjects in the active formulation group had successful improvement in IGA score from baseline to Week 12 compared with subjects in the vehicle control group (24.5% versus 7.3%; p=0.0070). These changes corresponded to LS mean percent changes from baseline of −44.5%, −67.0%, and −65.0%, respectively, for subjects in the Vehicle Gel group and −34.2%, −48.7%, and −53.1%, respectively, for subjects in the Vehicle Gel group. A statistically significant difference between treatment groups for change in inflammatory lesion count was observed only at Week 12 (p=0.0003 for the absolute change and p=0.0006 for the percent change).

Measures of sebum excretion showed small mean decreases in both treatment groups, with generally greater decreases for subjects treated with the active formulation than with the vehicle formulation. However, the variability of response was relatively large in both treatment groups for meaningful interpretation. Biomarker analysis showed no changes in lipid metabolism with in the active formulation group, either in relation to the vehicle or over time in treated subjects.

### Example 5

### Dose Range Studies

Dose ranges were determined in adult subjects with acne vulgaris on the face. The study was a randomized, vehicle controlled, parallel group study designed to assess the efficacy and safety of the active formulations comprising a compound of Formula (Ia) at a concentration of 7.5% BID, 7.5% QD, and 4.0% QD, respectively. The results were compared to those of vehicle formulation (BID or QD) on subjects with moderate to severe facial acne.

A total of 420 adult subjects were randomized to active formulations (7.5% BID, 7.5% QD, and 4.0% QD) and vehicle (BID and QD) in a 2:2:2:1:1 fashion. Study treatments continued for 12 weeks. Subjects returned to the study clinic at Weeks 1, 2 (phone call only), 4, 8 and 12 (study exit).

Safety was assessed through adverse events (AEs), local skin responses (LSRs, determined by the presence and severity of erythema, dryness, peeling, burning/stinging, and pruritus), laboratory tests (serum chemistry, and hematology), vital signs, and physical examinations.

Primary efficacy endpoints for this study were 1) the mean absolute change from baseline to Week 12 in inflammatory and noninflammatory lesion counts and 2) the proportion of subjects who achieved a 2-grade improvement in the IGA from baseline to Week 12. Absolute change from baseline to Week 12 in inflammatory and non-inflammatory lesion counts was analyzed using an analysis of covariance (ANCOVA) model with a factor of treatment and the respective baseline lesion count as a covariate. The proportion of subjects who are dichotomized to success (minimum 2-grade improvement from baseline in IGA score) at Week 12 was analyzed using a Cochran-Mantel-Haenszel (CMH) test. Exploratory analyses were conducted for linearity of a dose response for the proportion of subjects dichotomized to an IGA success.

A subset of study centers enrolled subjects for an assessment of PK. Blood was to be collected from approximately 10 subjects per treatment group. At the Day 1 and Week 8 visits, a predose blood sample was collected prior to the first application of study drug for the day; samples were then collected at 1, 2, 3, and 4 hours after application of study drug.

### Safety Results

The most common AEs reported during the study were nasopharyngitis, upper respiratory tract infection, and application site pruritus. Most AEs were mild or moderate in severity. Erythema was the most common LSR. Laboratory values, vital signs, and ECGs measured at the end of the study were generally consistent with baseline values, with no clinically significant trends.

### Efficacy Results

The results of the study showed that all three active treatment groups showed statistically significantly greater reductions in the absolute change in inflammatory lesion counts from baseline to Week 12 than the combined vehicle group. The LS mean changes in inflammatory lesion counts were −14.6 and −14.5, and −15.0 for the 4.0% QD, 7.5% QD, and 7.5% BID groups, respectively, compared with −10.7 for the combined vehicle QD group (P=0.011, P=0.014, and P=0.011, respectively). All 3 active treatment groups showed statistically significantly greater reductions in the absolute change in noninflammatory lesion counts from baseline to Week 12 than the combined vehicle group. The LS mean changes in noninflammatory lesion counts at Week 12 were −15.3, −13.4, and −17.5 for the 4.0% QD, 7.5% QD, and 7.5% BID groups, respectively, compared with −9.3 for the combined vehicle group (P=0.004, P=0.050, and P<0.011, respectively).

The 4.0% QD and 7.5% BID groups each had a statistically significantly greater proportion of subjects achieve a minimum 2-grade improvement (reduction) in IGA score from baseline at Week 12 compared with the combined vehicle group. The percentage of subjects achieving this endpoint was 21.6% in the 4.0% QD and 25.9% of subjects in the 7.5% BID group (compared with 9.8% in the combined vehicle QD group (P=0.024 and P=0.004, respectively).

Pharmacokinetic (PK) results, assessed in a subset of subjects, showed that plasma concentrations of the Compound of Formula (Ia) on Day 1 were undetectable for all but one subject, who had a plasma concentration of 0.304 ng/mL at one time point (2 hours post-dosing). Plasma concentrations at Week 8 were undetectable for all tested subjects. Plasma concentrations of TOFA on Day 1 were undetectable in most subjects, but detectable in a few subjects in each dose group, with values ranging from 0.101 to 1.02 ng/mL. Plasma concentrations of TOFA at Week 8 were undetectable for most subjects in the QD dose groups, but detectable in a few subjects, with values ranging from 0.100 to 0.299 ng/mL. In the 7.5% BID group, approximately half of the tested subjects had detectable TOFA levels at each time point, with mean values ranging from 0.156 to 0.340 ng/mL.

It is thus demonstrated that a dermatological formulation of Formula (Ia) at each of 3 dosing groups, 4.0% QD, 7.5% QD, and 7.5% BID, was well tolerated over a 12-week treatment period. Subjects treated in all three active treatment groups showed statistically significantly greater reductions in the absolute change in inflammatory lesion counts and noninflammatory lesion counts from baseline to Week 12 than the combined vehicle groups. The 4.0% QD and 7.5% BID groups each had a statistically significantly greater proportion of subjects achieve a minimum 2-grade improvement (reduction) in IGA score from baseline at Week 12 compared with the combined vehicle group.

All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

1. A drug product composition comprising a compound of Formula (I) 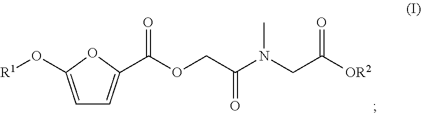
and
one or more impurities selected from the group consisting of: 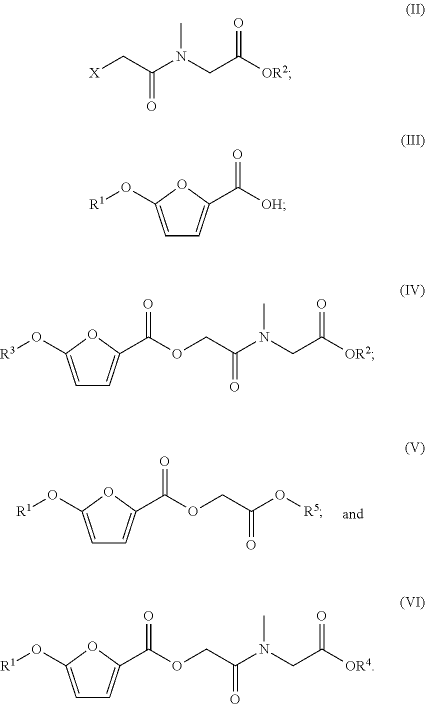
wherein,
R¹ is C₁₀₋₂₀ alkyl;
R² is C₁₋₄ alkyl;
R³ is C₁₀₋₂₀ alkyl, provided that R³ is not the same as R¹;
R⁴ is hydrogen, —(CH₂)C(O)N(CH₃)CH₂C(O)OR², or C₁₋₄ alkyl, provided that R⁴ is not the same as R²;
R⁵ is methyl or ethyl; and
X is halo,
and wherein, the one or more impurities together are no more than 3% w/w of the drug product composition.

2. The drug product composition of claim 1 wherein the compound of Formula (I) is represented by Formula (Ia)

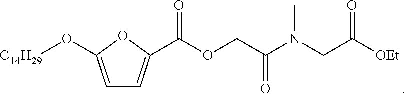

3. The drug product composition of claim 1 wherein R³ is —C₁₂H₂₅, —C₁₃H₂₇, —C₁₅H₃₁, —C₁₆H₃₃, or —C₁₈H₃₇.

4. The drug product composition of claim 1 wherein R⁴ is hydrogen or methyl.

5. The drug product composition of claim 1 wherein X is bromo or chloro.

6. The drug product composition of claim 1 wherein the one or more impurities represented by Formula (IV) are no more than 2% w/w of the drug product composition.

7. The drug product composition of claim 1 wherein the one or more impurities represented by Formula (II) are no more than 120 ppm of the drug product composition.

8. The drug product composition of claim 1 wherein the byproduct represented by Formula (VI), wherein R⁴ is hydrogen, is no more than 0.5% w/w of the drug product composition.

9. The drug product composition of claim 1 wherein the one or more impurities represented by Formula (V) are no more than 0.2% w/w of the drug product composition.

10. A dermatological formulation comprising:
a compound of Formula (I) 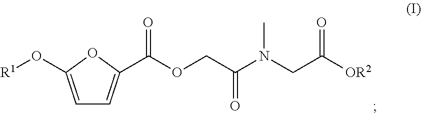
a dermatologically acceptable vehicle; and
one or more impurities selected from the group consisting of: 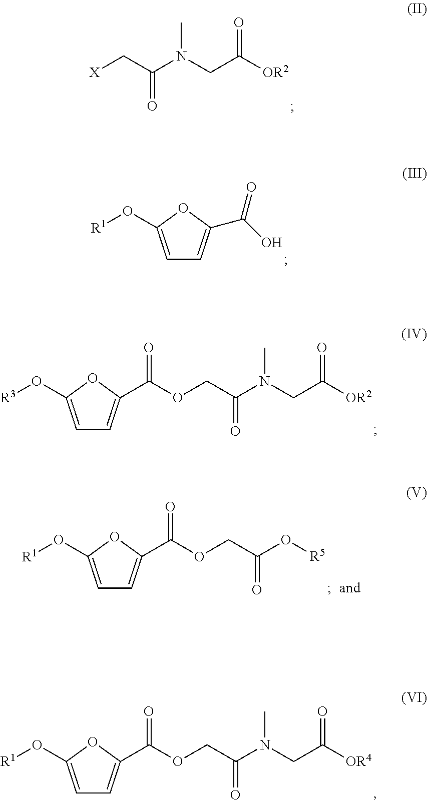
wherein,
R¹ is C₁₀₋₂₀ alkyl;
R² is C₁₋₄ alkyl;
R³ is C₁₀₋₂₀ alkyl, provided that R³ is not the same as R¹;
R⁴ is hydrogen, —(CH₂)C(O)N(CH₃)CH₂C(O)OR², or C₁₋₄ alkyl, provided that R⁴ is not the same as R²;
R⁵ is methyl or ethyl; and
X is halo, and wherein the one or more impurities together are no more than 3% w/w of the compound of Formula (I).

11. The dermatological formulation of claim 10 wherein the one or more impurities together are no more than 2% w/w of the compound of Formula (I).

12. The dermatological formulation of claim 10 wherein the compound of Formula (I) is

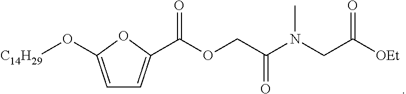

13. The dermatological formulation of claim 10 wherein R³ is —C₁₂H₂₅, —C₁₃H₂₇, —C₁₅H₃₁, —C₁₆H₃₃, or —C₁₈H₃₇.

14. The dermatological formulation of claim 10 wherein R⁴ is hydrogen or methyl.

15. The dermatological formulation of claim 10 wherein X is bromo or chloro.

16. The dermatological formulation of claim 10 wherein the one or more impurities represented by Formula (IV) are no more than 2% w/w of the compound of Formula (I).

17. The dermatological formulation of claim 10 wherein the one or more impurities represented by Formula (II) are no more than 120 ppm of the compound of Formula (I).

18. The dermatological formulation of claim 10 wherein the impurity represented by Formula (VI), wherein R⁴ is hydrogen, is no more than 0.5% w/w of the compound of Formula (I).

19. The dermatological formulation of claim 10 wherein the one or more impurities represented by Formula (V) are no more than 0.2% w/w of the compound of Formula (I).

20. A dermatological formulation comprising the drug product of claim 1 and a dermatologically acceptable excipient, wherein the compound of Formula (I) is present at a concentration of more than 1%, but no more than 7.5% (w/w) of the dermatological formulation.

21. The dermatological composition of claim 20 wherein the compound of Formula (I) has a structure represented by Formula (Ia).

22. A method for treating acne vulgaris comprising administering a dermatological formulation comprising a compound of Formula (Ia) topically to a subject in an area affected by acne vulgaris at least once daily, and wherein the compound of Formula (Ia) is present in the dermatological formulation at a concentration of 5% (w/w) or less and an impurity represented by Formula (IVa) is present at no more than 1% w/w of the compound of Formula (Ia).

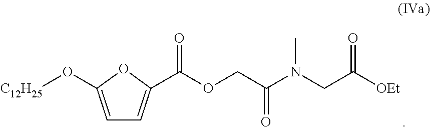

23. The method of claim 22 wherein the impurity represented by Formula (IVa) is present at no more than 0.1% w/w of the compound of Formula (Ia).

24. The method of claim 22 wherein the dermatological formulation is applied to the area affected by acne vulgaris twice daily.

25.-26. (canceled)

27. The drug product composition of claim 1 wherein the one or more impurities include: 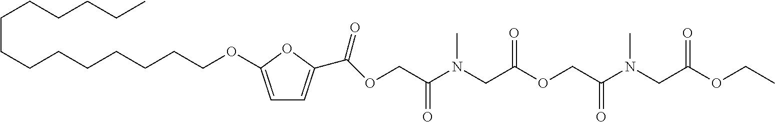 [SMILES: CCCCCCCCCCCCCCOc1ccc(C(=O)OCC(=O)N(C)CC(=O)OCC(=O)N(C)CC(=O)OCC)o1]
at an amount of no more than 0.5% w/w of the drug product composition.

28. The dermatological formulation of claim 10 wherein the one or more impurities include: 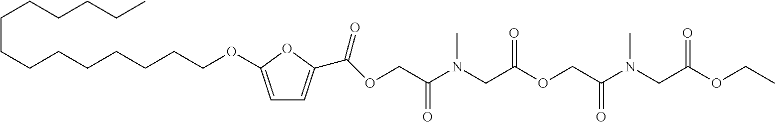 [SMILES: CCCCCCCCCCCCCCOc1ccc(C(=O)OCC(=O)N(C)CC(=O)OCC(=O)N(C)CC(=O)OCC)o1]
at an amount of no more than 0.5% w/w of the compound of Formula (I).
